(19) United States
(12) Patent Application Publication
(10) Pub. No.: US 2012/0265236 A1
(43) Pub. Date: Oct. 18, 2012

(54) CATHETER DEVICE

(75) Inventor: Matthias Wesselmann, Ruedlingen (CH)

(73) Assignee: BIOTRONIK AG, Bulach (CH)

(21) Appl. No.: 13/434,089

(22) Filed: Mar. 29, 2012

Related U.S. Application Data
(60) Provisional application No. 61/475,243, filed on Apr. 14, 2011.

(51) Int. Cl. A61M 29/02; A61M 29/00

(52) U.S. Cl. 606/194; 606/191

(57) ABSTRACT

A catheter device for expanding stenoses in the branching region of body vessels, comprising two catheter branches, the length of which is adjustable, and each of which has an outer distal rolling membrane, and which are interconnected at or near the distal end of a common catheter shaft.

### CROSS REFERENCE TO RELATED APPLICATIONS

This application is a nonprovisional application of U.S. provisional patent application Ser. No. 61/475,243, filed on Apr. 14, 2011; the contents of which are herein incorporated by reference in its entirety.

### TECHNICAL FIELD

The invention relates to a catheter device for expanding stenoses in body vessels.

### BACKGROUND

Such catheters have been known and used in clinical applications in diverse embodiments for decades, and have proven effective in various application situations. Variants referred to below as “hybrid catheters” represent a recent stage of development; they are known from U.S. Pat. No. 5,662,703, for example, and comprise a rolling membrane in addition to the actual balloon catheter. In the aforementioned document, the rolling membrane is used as a retaining device for a tubular, radially expandable stent which is delivered to the treatment site and deployed there by actuating the rolling membrane, in the slightly expanded state in particular. Document EP 0820259 B1 discloses such a hybrid catheter, the design of which is likewise adapted to the function of delivering a stent (in the radially compressed state in this case) to the application site thereof. Although the latter catheter device is designed primarily for use with self-expanding stents, it also comprises a dilatation catheter in one embodiment.

FIG. 1 shows a hybrid catheter 1 of the type under discussion here, in a schematic longitudinal cross-sectional view. Catheter 1 has an inner shaft 3 with a first proximal connector 3a for insertion of a guide wire 5, and a second proximal luer connector 3b for the application of pressure. An expandable balloon (inner balloon) 7 is installed on the distal end of inner shaft 3 and can be expanded using pressure present at connector 3b.

Inner shaft 3 is enclosed along the majority of its length by an outer shaft 9 which comprises, on the proximal end thereof, a low-friction seal insert 11 for sealing guidance on the outer circumference of inner shaft 3. A rolling membrane 13 is installed on the distal end of outer shaft 9, and is also fixedly connected to the distal end of the inner balloon at a connection point 13a, with pressure-tight separation therefrom. Rolling membrane 13, similar to inner balloon 7, is activated by pressure that is present at a proximal luer connector 9a of outer shaft 9. It should be mentioned that inner shaft 3 and outer shaft 9 comprise a common guide wire lumen 15 for guide wire 5.

A more recent, unpublished U.S. patent application belonging to the applicant also describes a new type of hybrid catheter characterized by a special design having an outer shaft, an inner shaft, and a central shaft, and a special axial positional relationship between the balloon and the rolling membrane. The use of such a catheter for expanding stenoses (independently of the use of a stent) is described in the latter document.

### SUMMARY

The problem addressed by the invention is that of providing an improved catheter device which offers advantages for expanding stenoses in branching regions in particular, and which can be handled easily and reliably.

This problem is solved by a catheter device having the features of claim 1. Advantageous developments of the idea according to the invention are the subject matter of the dependent claims. Furthermore, a method for operating the catheter device is provided.

According to a main idea of the invention, the catheter device comprises two catheter branches, each of which comprises an outer rolling membrane which is disposed substantially distally, and which are fixedly assigned relative to one another (before and after the stenosis is traversed and dilated). The fixed assignment of the two catheter branches in the resting state is brought about, according to a further aspect of the invention, by a common catheter shaft or fixedly connected catheter shafts, in which the two catheter branches are accommodated. According to a further aspect of the invention, the two catheter branches are suitably interconnected at or near the distal end, wherein the interconnection remains intact even when the function is deployed, and holds the two branches in a predetermined assignment relative to one another.

According to an embodiment of the invention, at least one of the two catheter branches comprises an inner balloon catheter part; preferably this applies for both catheter branches.

According to a further embodiment of the invention, the two catheter branches have different functional dimensions, in particular a different useful diameter of the respective rolling membrane and/or an inner balloon of the particular balloon catheter part. An advantageous embodiment of the latter embodiment is characterized by a non-confusable design of the two catheter branches, which is embodied in particular by guide wire lumen having a different diameter. This embodiment makes the handling of the catheter device provided easier and more reliable.

The two catheter branches are interconnected in a section of the circumference on the distal end of their outer shafts 7a. The connection can be established by welding, bonding outer shafts 7a, or by a commonly used outer shaft 7a. The result thereof is a certain stabilization of the entire device and otherwise functions in the sense of maintaining—as discussed above—a predetermined spatial assignment between the catheter branches even immediately before and/or during the execution of the function thereof.

According to a further embodiment of the invention, means are provided for affixing the axial position of inner balloon 7 of the balloon catheter part or each balloon catheter part with respect to the longitudinal axis of the catheter device. This is an undercut in the particular distal outer shaft of a catheter branch, in particular, which enables the inner balloon to easily unfold and thereby to become “wedged” in the outer shaft upon dilation. As an alternative or in addition thereto, the inner shaft can also be affixed axially on the proximal end using a pinch seal (of the Tuohy Borst type, for example). This function is particularly important since it allows the balloon lengths to be adapted in a flexible manner.

According to an embodiment of the invention the length of the two catheter branches is individually adjustable. Also the position of the two catheter branches in the artery is individually adjustable. Both these feasable adjustments render possible that only the stenosis in the branching region is treated.

According to a further embodiment of the invention, a coating of an active agent is provided on the circumference of the two catheter branches on the rolling membrane sections thereof. When the rolling membrane is unrolled, blood flow is stopped before the active agent is released. This reduces the loss of active agent, thereby allowing smaller quantities of the active agent to be dosed. Due, in particular, to the relatively high pressures applied by the rolling membrane on stenotic vascular sections, a coating of active agent can bring about an intensive transfer of active agent into the stenosis and, therefore, a strong therapeutic effect of a suitable active agent.

Advantages and useful features of the invention will also become apparent from the description of an embodiment with reference to the figures. In the figures:

### DESCRIPTION OF THE DRAWINGS

FIG. 1 shows a schematic view (simplified longitudinal cross section) of a hybrid catheter,

FIGS. 2A to 2D show the distal end of an embodiment of the invention in various operating states at a vascular branch having stenotic regions, and

FIG. 3 shows a modified embodiment of the balloon design of a catheter device according to the invention.

### DETAILED DESCRIPTION

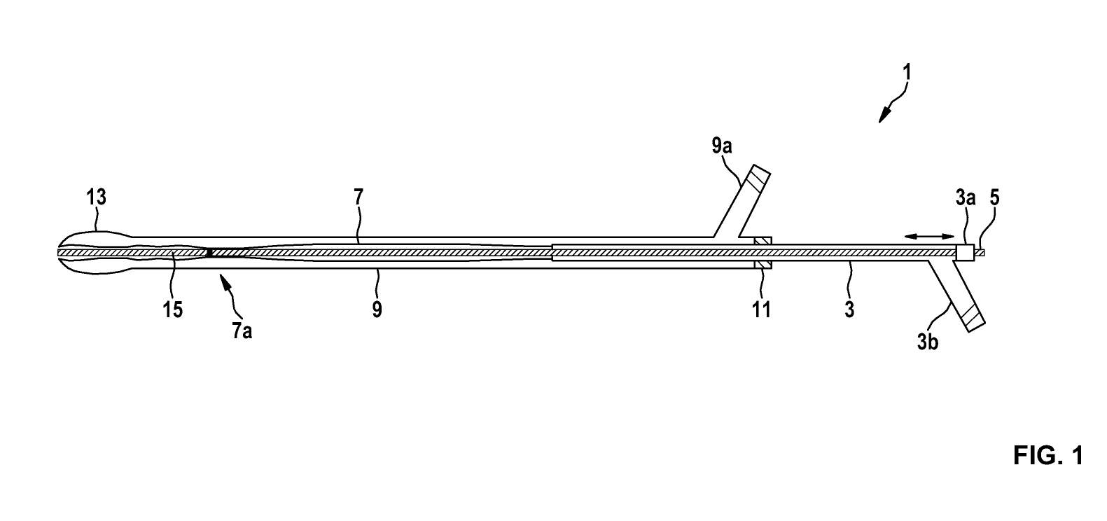

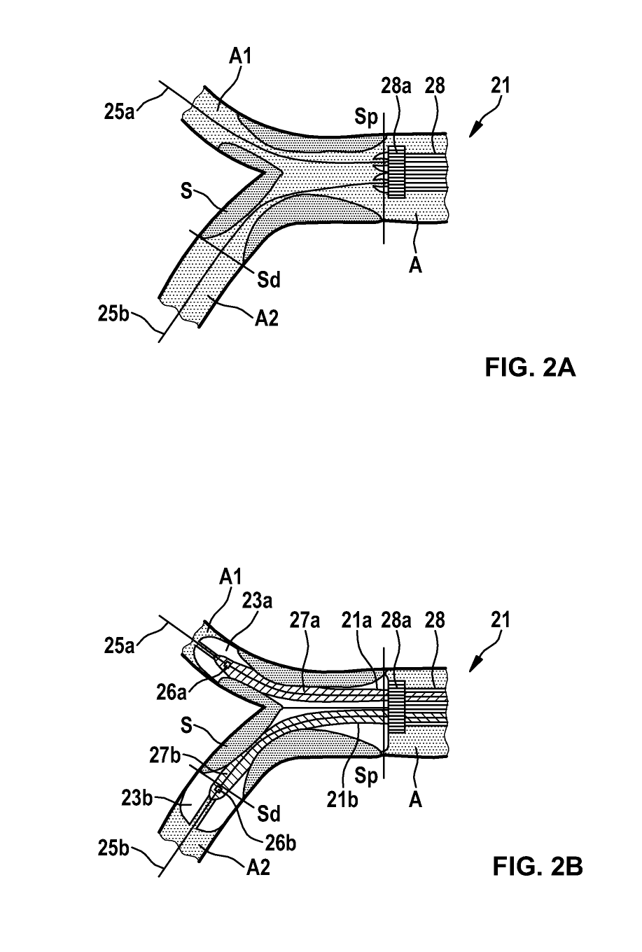

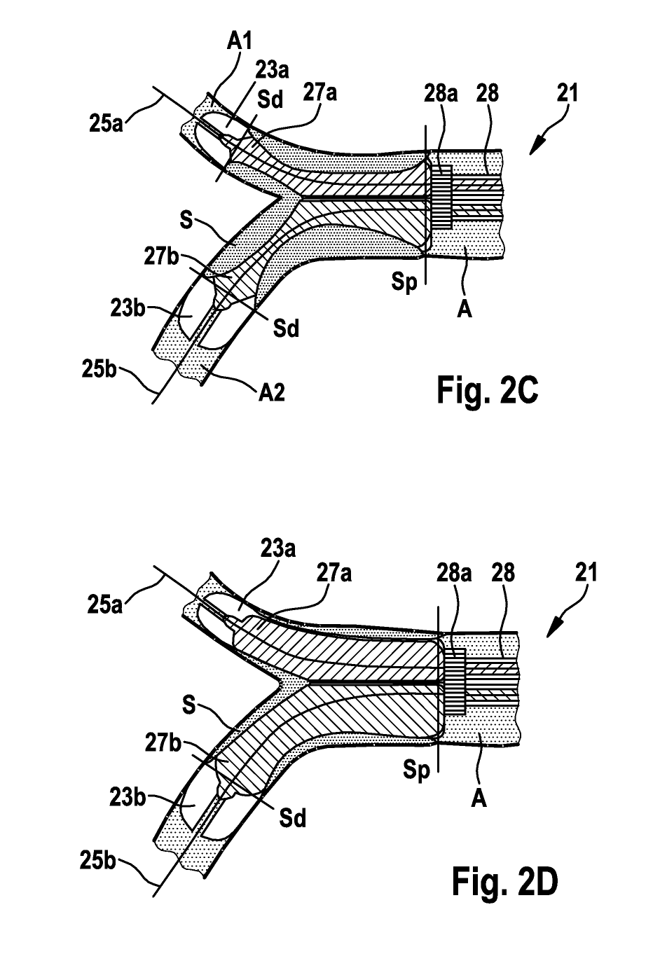

FIGS. 2A to 2D illustrate the mode of operation of a catheter device 21 according to an embodiment of the invention in various operating states. Only the distal end of the catheter device is shown in the figures. The further design thereof is clear to a person skilled in the art with consideration for the design of hybrid catheter 1 depicted in FIG. 1, which is essentially “doubled”, in particular, for the embodiment of the invention. Comparable parts or sections are labeled using the same reference numerals as in FIG. 1.

FIG. 2A shows a first operating state in which catheter device 21 is advanced by way of distal end thereof—marked with an x-ray marker 28a as the distal connector of a common catheter shaft 28—to proximal end Sp of a stenosis S in the branching region of an artery A. A first and a second guide wire 25a, 25b have already have already been slid out of the distal end of catheter device 21 and into branch A1 and A2, respectively, of artery A. Guide wires 25a and 25b have different diameters, of approximately 0.014″ and 0.018″, for example. The suitable position of the distal end of the outer shaft of catheter device 21 for the subsequent steps is determined by x-ray monitoring using x-ray marker 28a.

Pressure is now applied to the rolling membranes using (particular) luer connector 3b. This pressure causes the rolling membrane to unroll, thereby inducing a tensile force on the inner shaft on the distal end, which is proportional to the rolling membrane pressure. Simultaneously, the inner balloon is compressed further. Traversing the stenosis is greatly simplified
since there is no relative motion between the outside of the rolling membrane and the stenosis,
since the outside of the rolling membrane can touch the inside of the rolling membrane only on a line, thereby ensuring that the frictional force inside the rolling membrane remains minimal,
and since the inner balloon catheter and the wire provide additional pushability.

It must be ensured that the guide wire adheres to the inside of the rolling membrane in the inflated state of the rolling balloon, thereby enabling it to be advanced when passing through the stenosis. If there is a risk of distal perforation of the stenosis by the guide wire, the rolling membrane must be deflated briefly so that the guide wire can be pulled back.

FIG. 2B shows an operating state after both of the branches A1 and A2 of the stenotic artery A have been rolled through by extending the distal ends of a first catheter branch 21a and a second catheter branch 21b from common catheter shaft 28, positioned at the proximal end Sp of the stenosis S, along guide wire 25a and 25b, respectively, into arterial branches A1 and A2, respectively. Each catheter branch 21a, 21b has a rolling membrane 23a, 23b, respectively, on the distal end thereof, which have already been inflated and unrolled in the operating state shown in FIG. 2B. The two branches of the stenoses can be rolled through simultaneously or one after the other. The atraumatic rolling-through of the stenotic region is halted at respective distal end Sd of stenosis S in the respective branch by the operator affixing the inner balloon catheter at the proximal end thereof in a clamping device. Hence, the length of the two catheter branches is individually adjustable. In addition to the rolling membranes, each catheter branch 21a, 21b comprises an inner balloon 27a, 27b, respectively. In the state shown here, they are still in the non-expanded starting state.

The two catheter branches 21a, 21b differ in terms of their dimensions that are essential to function (referred to below as the functional dimensions), wherein FIG. 2B shows that the diameter of rolling membrane 23b, which has been placed in the other arterial branch A2, is greater than that of rolling membrane 23a in the other catheter branch 21a, which has been advanced into narrower artery A1. The non-confusable delivery into the catheter branch with the “correct” functional dimensions in the corresponding arterial branch is ensured in that the guide wire lumina of the two catheter branches also have different diameters such that guide wire 25b, with the greater diameter, can be advanced only into catheter branch 21b with the guide wire lumen having the larger diameter, thereby specifying the position thereof. The result is the correct path of guide wire 25a having the smaller diameter and, therefore, that of catheter branch 21a having the smaller functional dimensions.

To correctly determine the distal position of the catheter branches, each comprises an x-ray marker 26a and 26b (shown only in FIG. 2B). As soon as corresponding catheter branch 21a/b has rolled through the stenotic region, it is prevented from rolling further by being affixed on the proximal end of common catheter shaft 28. The rolling membrane must be deflated before the inner balloon is inflated.

As shown in FIG. 2C, the particular stenotic region is opened by the force of balloons 27a, 27b expanding, in the position of the catheter branches predetermined and held in the manner described above. To prevent balloons 27a, 27b from extending axially—which is undesired—along the particular longitudinal axis of the catheter branch due to displacement of the inner shaft, the catheter branch shafts can be affixed at the proximal catheter outlet using a Tuohy Borst seal, for example.

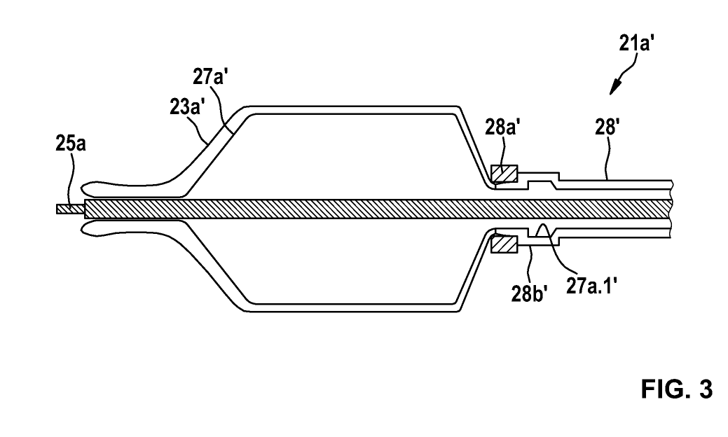

In addition, the inner balloon, as shown in FIG. 3 for one of the catheter branches as a modified embodiment 21a′, can be “anchored” on the distal end of catheter shaft 28′ by an undercut 28b′ in the outer shaft, since the dilatation forces are usually so great, at maximum pressure, that proximal affixation is insufficient. The inner shaft is loaded when pulled, and the outer shaft is loaded when pushed, and they respond accordingly with an extension of the inner shaft and a shortening of the outer shaft, and so the dilatable balloon length would be extended—which is undesirable—if countermeasures were not taken. The small counterballoon 27a.1′ forming in undercut 28b′ ensures that the forces on the inner balloon have axial equilibrium, thereby stabilizing the position thereof.

FIG. 2D shows an expanded state of the stenosis, i.e. a desired therapeutic result.

At the conclusion of the treatment, balloons 27a, 27b of catheter branches 21a, 21b are deflated, the connected dilatation pump (not shown) is released, and rolling membranes 23a, 23b are rolled back into common catheter shaft 28 under slight internal pressure. Finally, catheter device 27 and guide wires 25a, 25b are withdrawn from the treated vessel.

The embodiment of the invention is not limited to the above-described examples and emphasized aspects, but rather is possible in a large number of modifications that lie within the scope of handling by a person skilled in the art.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. A catheter device for expanding stenoses in the branching region of body vessels, comprising two catheter branches, each of which has an outer distal rolling membrane, and which are interconnected at or near a distal end of a common catheter shaft.

2. The catheter device according to claim 1, wherein at least one of the two catheter branches comprises an inner balloon catheter part.

3. The catheter device according to claim 2, wherein each of the two catheter branches comprises an inner balloon catheter part.

4. The catheter device according to claim 2, wherein the balloon of the inner balloon catheter part or each inner balloon catheter part comprises a balloon-end marker on or near the distal end thereof.

5. The catheter device according to claim 2, further comprising a means for positional affixation of the inner balloon of the balloon catheter part or each balloon catheter part on the longitudinal axis by way of at least one undercut in the outer shaft.

6. The catheter device according to claim 1, wherein the two catheter branches have a different useful diameter of the respective rolling membrane and/or an inner balloon of a balloon catheter part.

7. The catheter device according to claim 6, wherein the two catheter branches have a guide wire lumen with a different diameter to prevent confusion between the two.

8. The catheter device according to claim 1, wherein the two catheter branches are interconnected by a weld in a section of the circumference thereof.

9. The catheter device according to claim 1, wherein a length of the two catheter branches is individually adjustable.

10. The catheter device according to claim 1, wherein a position of the two catheter branches is individually adjustable.

11. The catheter device according to claim 1, wherein a coating of active agent and/or a coating of a sliding agent are/is provided internally on a circumference of both catheter branches on the rolling membrane.

12. A method of operating a catheter device according to claim 2, comprising the steps of:
advancing the catheter shaft to a beginning of the stenosis in the branching region of body vessels,
inflating the rolling membranes,
rolling through the stenotic regions in both vessels in parallel or sequentially, in an atraumatic manner, using the rolling membranes of the two catheter branches,
stopping the unrolling of the rolling membranes, so that a distal marker of the inner balloon has just passed through the stenotic region of the particular body vessel,
deflating both rolling membranes,
filling the balloons of the inner balloon catheter parts of both catheter branches to open the stenosis in the branching region of body vessels,
deflating the balloons of the inner balloon catheter parts,
inflating the rolling membranes and releasing the dilatation pump,
rolling the rolling membranes back into the catheter shaft, and
withdrawing the catheter shaft.
